(19) United States
(12) Patent Application Publication
(10) Pub. No.: US 2013/0245061 A1
(43) Pub. Date: Sep. 19, 2013

(54) PHARMACEUTICAL COMPOSITIONS

(71) Applicants: Yu Cao, Montville, NJ (US); Wolfgang Hackl, Loerach (DE); Ping Li, Basking Ridge, NJ (US); Shoufeng Li, Basking Ridge, NJ (US); Tahseen Mirza, Rockville, MD (US); Hartmut Zehender, Fulleren (FR); Jiahao Zhu, Whippany, NJ (US)

(72) Inventors: Yu Cao, Montville, NJ (US); Wolfgang Hackl, Loerach (DE); Ping Li, Basking Ridge, NJ (US); Shoufeng Li, Basking Ridge, NJ (US); Tahseen Mirza, Rockville, MD (US); Hartmut Zehender, Fulleren (FR); Jiahao Zhu, Whippany, NJ (US)

(73) Assignee: NOVARTIS AG, Basel (CH)

(21) Appl. No.: 13/989,250

(22) PCT Filed: Dec. 1, 2011
(86) PCT No.: PCT/US11/62837

§ 371 Date: May 23, 2013

Related U.S. Application Data
(60) Provisional application No. 61/419,538, filed on Dec. 3, 2010.
(60) Provisional application No. 61/436,324, filed on Jan. 26, 2011.

(51) Int. Cl. C07D 471/04

(52) U.S. Cl. CPC C07D 471/04
USPC 514/293; 264/118

(57) ABSTRACT

A pharmaceutical composition for the oral administration of a therapeutic compound of formula (I), comprising granules that comprise at least therapeutic compound of formula (I) or a tautomer thereof, or a pharmaceutically acceptable salt, or a hydrate or solvate thereof; at least one non-ionic surfactant that is Vitamin E-TPGS in an amount ranging from about 15 to about 80% by weight of the composition; and at least one a dissolution enhancing agent selected from polyethylene glycol, polyethylene oxide, and any combination of the foregoing; processes for making such pharmaceutical compositions; a kit comprising such pharmaceutical composition and the instructions for administration thereof; and related uses and methods of treatment.

### FIELD OF THE INVENTION

The present invention relates to a pharmaceutical composition for the oral administration of a therapeutic compound of formula (I), which comprises granules that comprise a therapeutic compound of formula (I) (see below), particularly 2-Methyl-2-[4-(3-methyl-2-oxo-8-quinolin-3-yl-2,3-dihydro-imidazo[4,5-c]quinolin-1-yl)-phenyl]-propionitrile or 8-(6-methoxy-pyridin-3-yl)-3-methyl-1-(4-piperazin-1-yl-3-trifluoromethyl-phenyl)-1,3-dihydro-imidazo[4,5-c]quinolin-2-one, or a tautomer thereof, or a pharmaceutically acceptable salt, or a hydrate or solvate thereof; at least one non-ionic surfactant that is Vitamin E-TPGS in an amount ranging from about 15 to about 80% by weight of the composition; and at least one a dissolution enhancing agent selected from polyethylene glycol, polyethylene oxide, and any combination of the foregoing. The present invention also relates to processes for making such pharmaceutical compositions.

### BACKGROUND OF THE INVENTION

In normal cells, the phosphatidylinositol-3-kinase (PI3K) is a regulator of multiple cellular functions, including protein synthesis and glucose metabolism, cell survival and growth, proliferation, cellular resilience and repair, cell migration, and angiogenesis. There is substantial evidence that in many tumors the PI3K signaling pathway is constitutively activated. Activation of the PI3K pathway via mutations in the catalytic subunit (PIK3CA) or inactivation of negative regulators (i.e., PTEN) results in constitutive signaling and oncogenicity. Deregulation of the PI3K pathway is established to be one of the most frequent occurrences in various human cancers, including but not limited to pancreatic cancer and breast cancer.

The specific imidazoquinoline derivative compounds of formula (I)

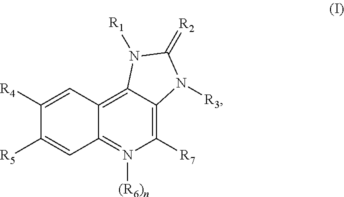

wherein R₁, R₂, R₃, R₄, R₅, n, R₆ and R₇ defined as set forth herein, or a tautomer thereof, or a pharmaceutically acceptable salt, or a hydrate or solvate thereof, are dual phosphatidylinositol-3-kinase (PI3K) and mammalian target of rapamycin (mTOR) inhibitors which may be used for the treatment of various proliferative disorders. These compounds and their preparation are disclosed in WO2006/122806. Such imidazoquinoline derivatives, such as 2-Methyl-2-[4-(3-methyl-2-oxo-8-quinolin-3-yl-2,3-dihydro-imidazo[4,5-c]quinolin-1-yl)-phenyl]-propionitrile or 8-(6-methoxy-pyridin-3-yl)-3-methyl-1-(4-piperazin-1-yl-3-trifluoromethyl-phenyl)-1,3-dihydro-imidazo[4,5-c]quinolin-2-one and pharmaceutically acceptable salts thereof, are proven to be effective dual PI3K/mTOR inhibitors, e.g., WO2006/122806, WO2008/103636 and Maira et al, Mol. Cancer. Ther., 7(7): 1851-1863 (July 2008) which display broad activity against a large panel of cultured human cancer cell lines.

There is a need to formulate the specific imidazoquinoline derivative compounds of formula (I), particularly 2-Methyl-2-[4-(3-methyl-2-oxo-8-quinolin-3-yl-2,3-dihydro-imidazo[4,5-c]quinolin-1-yl)-phenyl]-propionitrile or 8-(6-methoxy-pyridin-3-yl)-3-methyl-1-(4-piperazin-1-yl-3-trifluoromethyl-phenyl)-1,3-dihydro-imidazo[4,5-c]quinolin-2-one, or a tautomer or a pharmaceutically acceptable salt or a hydrate or solvate thereof, into pharmaceutical compositions, especially solid oral dosage forms, such that the therapeutic benefits of the compounds may be delivered to a patient in need thereof. Posing as a challenge to resolving this need is the physiochemical properties of such therapeutic compounds. The compounds of formula (I), particularly 2-Methyl-2-[4-(3-methyl-2-oxo-8-quinolin-3-yl-2,3-dihydro-imidazo[4,5-c]quinolin-1-yl)-phenyl]-propionitrile and its pharmaceutically acceptable salt, have low permeability and strong pH-dependent solubility (approximately 0% solubility when the pH is higher than 3). These compounds are difficult to formulate and deliver (i.e., made bioavailable when ingested orally) and demonstrate a tendency to dissolve in the acidic conditions in the stomach and subsequently precipitate in the intestines under neutral pH conditions.

An objective of the present invention is to provide a pharmaceutical composition that can improve the dissolution and absorption of the therapeutic compound upon administration to a subject in need thereof. It is further an objective of the present invention to provide a pharmaceutical composition in the form of an oral dosage form that may be ingested by a patient.

Surprisingly, it has been discovered that the pharmaceutical compositions of the present invention unexpectedly achieved significant improvement in the dissolution and the absorption of the therapeutic compound upon administration of the therapeutic compound to a subject in need thereof. It has further been discovered that the pharmaceutical compositions of the present invention can be formulated to have supersaturation upon acid-to-neutral pH transition in the gastrointestinal tract and into an oral dosage form which can be safely ingested by a subject in need thereof.

### SUMMARY OF THE INVENTION

The present invention relates to a pharmaceutical composition for the oral administration of a therapeutic compound of formula (I), as defined below, which comprises granules that comprise a therapeutic compound of formula (I), at least one non-ionic surfactant that is Vitamin E TPGS in an amount ranging from about 15 to 80% by weight of the composition and at least one dissolution enhancing agent selected from polyethylene glycol, polyethylene oxide, and any combination of the foregoing. The pharmaceutical composition may optionally further comprise pharmaceutically acceptable disintegrants, binders, lubricants, glidants, fillers, diluents, colorants, flavours or preservatives.

In a preferred embodiment of the present invention, the pharmaceutical composition of the present invention is a supersaturation system.

The therapeutic compound of formula (I), particularly COMPOUND A or COMPOUND B, is present in an amount ranging from about 1 to about 99%, preferably from about 30 to about 60%, and even more preferably from about 35% to about 60%.

The Vitamin E TPGS is present in the pharmaceutical composition in an amount ranging from about 15 to about 80% by weight of the composition, e.g., from about 15 to about 45%, from about 15 to about 35%, or from about 30% to 45%.

The dissolution enhancing agent is present in the pharmaceutical composition in an amount ranging from about 1 to about 15% by weight of the composition, e.g., from about 1% to about 10%, e.g., from about 4% to about 8%, e.g. from about 4% to about 7%.

The present invention further relates to a process for making a pharmaceutical composition of the present invention by forming said granules by melt granulation and then formulating said granules into an oral dosage form.

The present invention further relates to an oral dosage form comprising the pharmaceutical composition of the present invention.

The present invention further relates to the use of the pharmaceutical composition of the present invention for the treatment of a proliferative diseases.

The present invention further relates to the use of the pharmaceutical composition of the present invention for the treatment of a mTOR kinase dependent disease.

The present invention further relates to kit comprising (a) an oral pharmaceutical composition which comprises granules that comprise a therapeutic compound of formula (I), at least one non-ionic surfactant that is Vitamin E TPGS in an amount ranging from about 15 to 80% by weight of the composition and at least one dissolution enhancing agent selected from polyethylene glycol, polyethylene oxide, and any combination of the foregoing, and (b) written instructions, wherein such written instructions provide that such oral pharmaceutical composition may be taken between thirty minutes prior to the consumption of food until about two hours after the consumption of food. Preferably, said written instructions provide that such oral pharmaceutical composition may be taken immediately to about thirty minutes after the consumption of food.

The present invention further relates to the use of the pharmaceutical composition of the present invention for the treatment of a proliferative disease or a mTOR kinase dependent disease comprising administering to a subject in need thereof a pharmaceutical composition which comprises granules that comprise a therapeutic compound of formula (I), at least one non-ionic surfactant that is Vitamin E TPGS in an amount ranging from about 15 to 80% by weight of the composition and at least one dissolution enhancing agent selected from polyethylene glycol, polyethylene oxide, and any combination of the foregoing, with food, wherein the administration of such pharmaceutical composition with food results in an increase in the bioavailability of the compound of formula (I) as compared to administration of the pharmaceutical composition to a subject in the fasted stated.

The present invention further relates to the use of the pharmaceutical composition of the present invention for the treatment of a proliferative disease or a mTOR kinase dependent disease comprising administering to a subject in need thereof a pharmaceutical composition which comprises granules that comprise a therapeutic compound of formula (I), at least one non-ionic surfactant that is Vitamin E TPGS in an amount ranging from about 15 to 80% by weight of the composition and at least one dissolution enhancing agent selected from polyethylene glycol, polyethylene oxide, and any combination of the foregoing, with food, wherein the administration of such pharmaceutical composition with food results in an increase in the bioavailability of the compound of formula (I) as compared to administration of the compound of formula (I) to a subject without food.

The present invention further relates to a method of treatment of a proliferative disease or a mTOR kinase dependent disease comprising administering to a subject in need thereof a pharmaceutical composition of the present invention comprising a therapeutically effective amount of the compound of formula (I) with food, wherein the administration of such pharmaceutical composition with food results in an increase in the bioavailability of the compound of formula (I) as compared to administration of the pharmaceutical composition to a subject without food.

The present invention further relates to a method of treatment of a proliferative disease or a mTOR kinase dependent disease comprising administering to a subject in need thereof a pharmaceutical composition of the present invention comprising a therapeutically effective amount of the compound of formula (I) with food, wherein the administration of such pharmaceutical composition with food results in an increase in the bioavailability of the compound of formula (I) as compared to administration of the compound of formula (I) to a subject without food.

### BRIEF DESCRIPTION OF THE DRAWINGS

The accompanying drawings, which are incorporated in and constitutes a part of the specification, illustrates exemplary embodiments of the present invention.

FIG. 1 shows a chart showing the dissolution profile for six different pharmaceutical compositions in capsule dosage forms. Each capsule included Compound A and the non-ionic surfactant Vitamin E TPGS in a ratio of 2:2. Four of the six capsules additionally include either 25 mg, 45 mg, 100 mg or 50 mg of PEG3350, and the capsule including 50 mg of PEG3350 additionally included 50 mg of crospovidone. The capsules are placed in 900 mL of 0.1N HCl (pH 1.2) using USP Apparatus II rotating at 100 rpm and at 37° C. The chart shows that the capsule containing both Vitamin E TPGS and PEG3350 achieves faster dissolution than the comparable capsule containing Vitamin E TPGS alone.

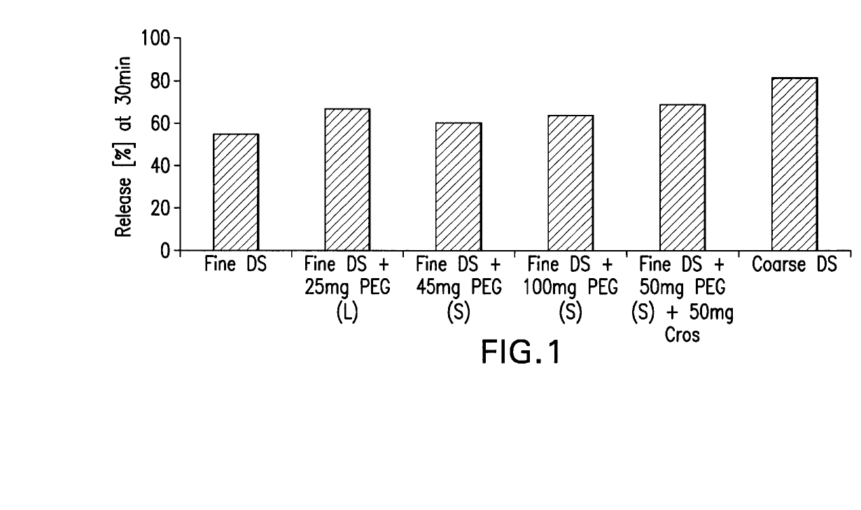

FIG. 2 shows a graph showing the results from the 2-Step Dissolution Test conducted to compare the dissolution profile for Compound A alone, Formulation Variant 1 and Formulation Variant 2 as described herein.

Capsules containing either Compound A alone, Formulation Variant 1 or Variant 2 are placed in 900 mL of pH 2 HCl and then stirred in a USP Apparatus II rotating at 50 rpm for 10 to 100 minutes (with fast stir from about minute 60 to minute 90). At approximately the 100 minute time point, this medium is adjusted to pH 6.8 and to a volume of 1000 ml and then stirred in a USP Apparatus II rotating at 50 rpm. Samples are filtered using a 100 μm Varian full flow filter and analyzed by UV.

FIG. 3 shows graphs showing the preliminary results from the study of patients having metastatic/advanced solid tumors and treated with 800 mg of Compound A. Patients were treated with either a SDS capsule or SDS sachet formulated in accordance with the present invention. The graphs show that the variability in total mean drug exposure for the SDS sachet is less than the variability seen for the SDS capsule.

### DETAILED DESCRIPTION OF THE INVENTION

The present invention relates to a pharmaceutical composition for the oral administration of a therapeutic compound of formula (I) which comprises granules that comprise a therapeutic compound of formula (I), at least one non-ionic surfactant that is Vitamin E TPGS in an amount ranging from about 15 to 80% by weight of the composition and at least one dissolution enhancing agent selected from polyethylene glycol, polyethylene oxide, and any combination of the foregoing. The pharmaceutical composition may optionally further comprise pharmaceutically acceptable disintegrants, binders, lubricants, glidants, fillers, diluents, colorants, flavours and preservatives.

As used herein the term “pharmaceutical composition” means a physical mixture containing a therapeutic compound to be administered to a mammal, e.g., a human in order to prevent, treat or control a particular disease or condition affecting the mammal. The term “pharmaceutical composition” as used herein, for example, also encompasses an intimate physical mixture formed at high temperature and pressure.

As used herein the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

As used herein the term “therapeutic compound” means any compound, substance, drug, medicament, or active ingredient having a therapeutic or pharmacological effect, and which is suitable for administration to a mammal, e.g., a human, in a composition that is particularly suitable for oral administration. Particularly useful as therapeutic compounds in the present invention are 2-methyl-2-[4-(3-methyl-2-oxo-8-quinolin-3-yl-2,3-dihydro-imidazo[4,5-c]quinolin-1-yl)-phenyl]-propionitrile and its monotosylate salt (COMPOUND A) and 8-(6-methoxy-pyridin-3-yl)-3-methyl-1-(4-piperazin-1-yl-3-trifluoromethyl-phenyl)-1,3-dihydro-imidazo[4,5-c]quinolin-2-one (COMPOUND B) and pharmaceutically acceptable salts thereof.

As used herein, the term “therapeutically effective amount” refers to an amount or concentration which is effective in reducing, eliminating, treating, preventing or controlling the symptoms of a disease or condition affecting a mammal. The term “controlling” is intended to refer to all processes wherein there may be slowing, interrupting, arresting or stopping of the progression of the diseases and conditions affecting the mammal. However, “controlling” does not necessarily indicate a total elimination of all disease and condition symptoms.

As used herein, the term “immediate release” refers to the rapid release of the majority of the therapeutic compound, e.g., greater than about 50%, about 60%, about 70%, about 80%, or about 90%, within a relatively short time, e.g., within 100 minutes, 60 minutes, 40 minutes, or 30 minutes after oral ingestion. The particular immediate release conditions for a specific therapeutic compound will be recognized or known by one of ordinary skill in the art.

As used herein the term “treatment” includes prophylactic (preventive) and therapeutic treatment as well as the delay of progression of a disease or disorder. The term “delay of progression” as used herein means administration of the pharmaceutical composition to patients being in a pre-stage or in an early phase of the proliferative disease to be treated, in which patients for example a pre-form of the corresponding disease is diagnosed or which patients are in a condition, e.g. during a medical treatment or a condition resulting from an accident, under which it is likely that a corresponding disease will develop.

As used herein, the term “administered with food” refers to, for example, any food product, solid or liquid, with caloric content. Preferably the food is a solid food with sufficient bulk and fat content that it is not rapidly dissolved and absorbed in the stomach. The dosage of the compound of formula (I) may be administered to a subject, for example, between thirty (30) minutes prior to eating food to about two (2) hours after consumption. Preferably, administration of a compound of formula (I) may occur immediately after consuming food up to about thirty (30) minutes after consumption.

As used herein, the term “without food” or “fasted” refers to, for example, the condition of not having consumed solid food for about or greater than one (1) hour prior to until about or greater than two (2) hours after such consumption.

In a preferred embodiment of the present invention, the pharmaceutical composition of the present invention is a supersaturation system. The term “supersaturated system” refers to a system which has a greater concentration of the active ingredient compound in solution than would exist at equilibrium.

WO2006/122806 describes imidazoquinoline derivatives, which have been described to inhibit the activity of lipid kinases, such as PI3-kinases. Specific imidazoquinoline derivatives which are suitable for the present invention, their preparation and suitable pharmaceutical compositions containing the same are described in WO2006/122806 and include compounds of formula (I)

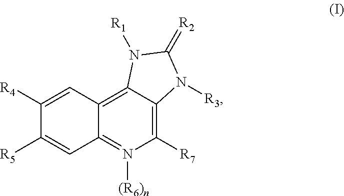

wherein
R₁ is naphthyl or phenyl wherein said phenyl is substituted by one or two substituents independently selected from the group consisting of Halogen; lower alkyl unsubstituted or substituted by halogen, cyano, imidazolyl or triazolyl; cycloalkyl; amino substituted by one or two substituents independently selected from the group consisting of lower alkyl, lower alkyl sulfonyl, lower alkoxy and lower alkoxy lower alkylamino; piperazinyl unsubstituted or substituted by one or two substituents independently selected from the group consisting of lower alkyl and lower alkyl sulfonyl; 2-oxo-pyrrolidinyl; lower alkoxy lower alkyl; imidazolyl; pyrazolyl; and triazolyl;

### R₂ is O or S;

R₃ is lower alkyl;
R₄ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen; quinoxalinyl; or phenyl substituted with alkoxy
R₅ is hydrogen or halogen;
n is 0 or 1;
R₆ is oxido;
with the proviso that if n=1, the N-atom bearing the radical R₆ has a positive charge;
R₇ is hydrogen or amino;
or a tautomer thereof, or a pharmaceutically acceptable salt, or a hydrate or solvate thereof.

The radicals and symbols as used in the definition of a compound of formula (I) have the meanings as disclosed in WO2006/122806 which publication is hereby incorporated into the present application by reference.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

In a preferred embodiment, “alkyl” has up to a maximum of 12 carbon atoms and is especially lower alkyl.

“Lower alkyl” is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or preferably methyl.

“Cycloalkyl” is preferably cycloalkyl with from and including 3 up to and including 6 carbon atoms in the ring; cycloalkyl is preferably cyclopropyl, cyclobutyl, cyclopentyl or cyclohexyl.

Alkyl which is substituted by halogen is preferably perfluoro alkyl such as trifluoromethyl.

Halogen is especially fluorine, chlorine, bromine, or iodine, especially fluorine, chlorine, or bromine.

In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient.

Salts are formed, for example, as acid addition salts, preferably with organic or inorganic acids, from compounds of formula (I) with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, malonic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 4-toluenesulfonic acid, 2-naphthalenesulfonic acid, 1,5-naphthalene-disulfonic acid, 2- or 3-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid. Preferably, the salt formed is the 4-toluenesulfonic acid.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

A preferred compound of the present invention is a compound which is specifically described in WO2006/122806. A very preferred compound of the present invention is 2-methyl-2-[4-(3-methyl-2-oxo-8-quinolin-3-yl-2,3-dihydro-imidazo[4,5-c]quinolin-1-yl)-phenyl]-propionitrile and its monotosylate salt (COMPOUND A). The synthesis of 2-methyl-2-[4-(3-methyl-2-oxo-8-quinolin-3-yl-2,3-dihydro-imidazo[4,5-c]quinolin-1-yl)-phenyl]-propionitrile and its monotosylate salt are for instance described in WO2006/122806, which is hereby incorporated in its entirety hereto, as Example 7 and Example 152-3 respectively. Another very preferred compound of the present invention is 8-(6-methoxy-pyridin-3-yl)-3-methyl-1-(4-piperazin-1-yl-3-trifluoromethyl-phenyl)-1,3-dihydro-imidazo[4,5-c]quinolin-2-one (COMPOUND B). The synthesis of 8-(6-methoxy-pyridin-3-yl)-3-methyl-1-(4-piperazin-1-yl-3-trifluoromethyl-phenyl)-1,3-dihydro-imidazo[4,5-c]quinolin-2-one is for instance described in WO2006/122806 as Example 86.

The therapeutic compound(s) is present in the pharmaceutical compositions of the present in a therapeutically effective amount or concentration. Such a therapeutically effective amount or concentration is known to a physician, clinician or veterinarian of ordinary skill in the art as the amount or concentration varies with the therapeutic composition being used and the indication which is addressed. The therapeutically effective amount or concentration of the therapeutic compound further depends upon a variety of additional factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy requires a regimen based on the kinetics of the drug's availability to target sites. This involves a consideration of the distribution, equilibrium, and elimination of the therapeutic compound.

For example, the dose of the therapeutic compound of formula (I) or a pharmaceutically acceptable salt thereof for a person of approximately 70 kg body weight will be in the range from about 3 mg to approximately 5 g, from approximately 10 mg to approximately 1.5 g, preferably from approximately 100 mg to about 1600 mg per person, preferably from approximately 800 mg to about 1600 mg per person, preferably from about 800 mg to about 1400 mg per person, preferably from about 100 mg to about 1000 mg per person per day, or about 200 mg to about 400 mg per person per day, divided preferably into 1 to 3 single doses which may, for example, be of the same size. Usually, children receive half of the adult dose.

In one embodiment, the therapeutic compound of formula (I) in the pharmaceutical composition is crystalline. Preferably, the pharmaceutical composition includes a crystalline form of COMPOUND A.

In accordance with the present invention, the therapeutic compound of formula (I), particularly COMPOUND A or COMPOUND B, is present in an amount ranging from about 1 to about 99%, preferably from about 30 to about 60%, and even more preferably from about 35% to about 60%.

The non-ionic surfactant Vitamin E-TPGS (water soluble D-alpha-tocopheryl polyethylene glycol succinate) is an amphipathic excipient which is a water soluble derivative of natural-source vitamin E. These include, e.g. those with a polymerisation number of approximately 1000, which is commercially available from Eastman Fine Chemicals Kingsport, Tex., USA. In addition to serving as a source of water-soluble vitamin E, vitamin E TPGS has been suggested for use as a hydrophilic non-ionic surfactant that can be used emulsifier, solubilizer, and bioavailability enhancer. The non-ionic surfactant Vitamin E-TPGS used in the present invention is believed to assist in the reduction or inhibition of the precipitation of the compound of formula (I).

Vitamin E TPGS, or PEGylated vitamin E, is a vitamin E derivative in which polyethylene glycol subunits are attached by a succinic acid diester at the ring hydroxyl of the vitamin E molecule. Vitamin E TPGS. Various chemical derivatives of vitamin E TPGS including ester and ether linkages of various chemical moieties are included within the definition of vitamin E TPGS.

Particularly preferred are D-alpha-tocopheryl polyethylene glycol succinate (Vitamin E TPGS) derivatives with PEG molecular weights between about 500 and 6000 Da. In a preferred embodiment, the Vitamin E TPGS used in accordance with the present invention is D-alpha-tocopheryl polyethylene glycol 1000 succinate (Vitamin E TPGS, tocopherlosan).

In accordance with the present invention, the Vitamin E TPGS is present in the pharmaceutical composition in an amount ranging from about 15 to about 80% by weight of the composition, e.g., from about 15 to about 45%, from about 15 to about 35%, or from about 30% to 45%. In one embodiment, the Vitamin E TPGS is present in the composition from about 15 to about 35% to produce a pharmaceutical composition having substantially uniform particle size distribution.

The dissolution enhancing agent of the present invention is selected from the group consisting of polyethylene glycol (PEG), polyethylene oxide, and any combination of the foregoing.

In the preferred embodiment, the dissolution enhancing agent is PEG which is the polymer of ethylene oxide that conforms generally to the formula H(OCH₂CH₂)ₙOH in which n represents the average molecular weight of the polymer.

The types of PEG useful in the present invention can be categorized by its state of matter, i.e., whether the substance exists in a solid or liquid form at room temperature and pressure. As used herein, “liquid PEG” refers to PEG having a molecular weight such that the substance is in a liquid or semi-liquid state at room temperature and pressure. For example, PEG having a molecular weight ranging between 100 and below 950 are liquid PEG. Such PEGs include, but are not limited to PEG 200, PEG 300, PEG 400, PEG 600 and PEG 800.

As used herein, “solid PEG” refers to PEG having a molecular weight such that the substance is in a solid state at room temperature and pressure. For example, PEG having a molecular weight ranging between 950 and 10,000 is a solid PEG. Such PEGs include, but are not limited to PEG 1000, PEG 1550, PEG 2000, PEG 3000, PEG 3350, PEG 4000 or PEG 8000. Particularly useful solid PEGs are those having a molecular weight between 1,450 and 8,000. Especially useful as a solid PEG are PEG 1450, PEG 3350, PEG 4000, PEG 8000, derivatives thereof and mixtures thereof. PEGs of various molecular weights are commercially-available as the CARBOWAX SENTRY series from Dow Chemicals (Danbury, Conn.).

Polyethylene oxide (“PEO”) which has an identical structure to PEG but for chain length and end groups are also suitable for use in the present invention. Various grades of PEO are commercially available as POLYOX from Dow Chemicals. PEO, for example, has a molecular weight ranging from about 100,000 to 7,000,000. The dissolution enhancing agent in the present invention can comprise PEG, PEO, and any combinations of the foregoing.

In the preferred embodiment, the dissolution enhancing agent is a solid PEG. Most preferred is a solid PEG that is PEG3350.

In an alternative exemplary embodiment, the dissolution enhancing agent of the carrier consists of a single dissolution enhancing agent, e.g., a solid PEG, e.g., PEG 1450, PEG 3350, PEG 4000 and PEG 8000. For example, if the pharmaceutical composition comprises PEG3350, the pharmaceutical composition would contain no other dissolution enhancing agent.

In yet another alternative exemplary embodiment, the dissolution enhancing agent of the carrier consists of a mixture of solid PEGs. For example, the dissolution enhancing agent comprises PEG 1450, PEG 3350, PEG 4000, PEG 8000, derivatives thereof and any combinations and mixtures thereof.

In accordance with the present invention, the dissolution enhancing agent is present in the pharmaceutical composition in an amount ranging from about 1 to about 15% by weight of the composition, e.g., from about 1% to about 10%, e.g., from about 4% to about 8%, e.g. from about 4% to about 7%.

In a preferred embodiment, the pharmaceutical composition is an immediate-release composition.

The present invention further relates to a process for making a pharmaceutical composition of the present invention by forming said granules by melt granulation and then formulating said granules into an oral dosage form.

As used herein, the term “melt granulation” refers to the following compounding process that comprises the steps of:

(a) forming a mixture of a therapeutic compound of formula (I) with at least one non-ionic surfactant that is Vitamin E TPGS, and at least one dissolution enhancing agent selected from the group consisting of polyethylene glycol (PEG), polyethylene oxide, and any combination of the foregoing,

(b) granulating the mixture using an extruder or other suitable equipment, for example a jacketed high shear mixer, while heating the mixture to a temperature above the softening temperature of the non-ionic surfactant that is Vitamin E TPGS; as used herein, the “softening temperature” refers to the temperature at which the non-ionic surfactant experiences a change in the rate of viscosity decrease as a function of temperature; and

(c) cooling the granules to room temperature, for example, at a controlled rate.

The heating and mixing of the compound of formula (I) with at least surfactant that is Vitamin E TPGS, and at least one dissolution enhancing agent selected from the group consisting of polyethylene glycol (PEG), polyethylene oxide, and any combination of the foregoing to form melt granulated granules may be accomplished, e.g., by the use of fluidized bed granulator or a vessel supplied with high-shear mixing means. The Vitamin E TPGS is present in the pharmaceutical composition from about 15 to about 80% by weight of the composition, e.g., from about 15 to about 45%, from about 15 to about 35%, or from about 30% to 45%. The dissolution enhancing agent is present in the pharmaceutical composition from about 1 to about 15% by weight of the composition, e.g., from about 1% to about 10%, e.g., from about 4% to about 8%, e.g. from about 4% to about 7%. The compound of formula (I), particularly COMPOUND A or COMPOUND B, may be present in an amount from about 1 to about 99%, preferably from about 30 to about 60%, and even more preferably from about 35% to about 60%.

Useful for effecting the melt granulation process is an extruder. In general, an extruder includes a rotating screw(s) within a stationary barrel with a die located at one end of the barrel. Along the entire length of the screw, distributive mixing of the materials (e.g., the therapeutic compound, release retarding material, and any other needed excipients) is provided by the rotation of the screw(s) within the barrel. Conceptually, the extruder can be divided into at least three sections: a feeding section; a heating section and a metering section. In the feeding section, the raw materials are fed into the extruder, e.g. from a hopper. The raw materials can be directly added to the hopper without the need of a solvent. In the heating section, the raw materials are heated to a temperature greater than the softening temperature of the release retarding material. After the heating section is a metering section in which the mixed materials are extruded through a die into a particular shape, e.g., granules or noodles, and further milled to form granules. Types of extruders particularly useful in the present invention are single-, twin- and multi-screw extruders, optionally configured with kneading paddles. Any type of mill as known by one of ordinary skill may be used in the present invention, for example a Frewitt hammer mill using 2 mm screen with a rate of 2000 rpm.

To make the pharmaceutical compound of the present invention, the therapeutic compound of formula (I) and the non-ionic surfactant Vitamin E TPGS are blended in a ratio in the range about 1:10 to about 10:1, e.g., 1:1, 1:2, 1:9, 2:6, or 3:2. Preferably, the ratio is in the range of about 1:1 or 1:2.

Mixing of the components can take place during or after heating. For example, the components may be either mixed first and then melted or simultaneously mixed and melted.

In one embodiment, the process of the present invention comprises the steps of (a) combining or mixing the total amount of the therapeutic compound of formula (I), the non-ionic surfactant Vitamin E TPGS, and the dissolution enhancing agent selected from the group consisting of polyethylene glycol (PEG), polyethylene oxide, and any combination of the foregoing, (b) granulating the mixture using an extruder or other suitable equipment, for example a jacketed high shear mixer, while heating the mixture to a product temperature below the melting point (about 38° C. to about 41° C.) of the Vitamin E TPGS; and (c) cooling the granules to room temperature, for example, at a controlled rate.

In a further embodiment, the process of the present invention comprises the steps of (a) dividing the total amount of the non-ionic surfactant Vitamin E TPGS into a first portion and a second portion, (a) combining or mixing the total amount of the therapeutic compound of formula (I), the first portion of the non-ionic surfactant Vitamin E TPGS, and the total amount of the dissolution enhancing agent selected from the group consisting of polyethylene glycol (PEG), polyethylene oxide, and any combination of the foregoing, (b) granulating the mixture using an extruder or other suitable equipment, for example a jacketed high shear mixer, while slowly adding the second portion of the non-ionic surfactant Vitamin E TPG to the mixture and maintaining a product temperature below about 38° C., and (c) cooling the granules to room temperature, for example, at a controlled rate. One of ordinary skill in the art will be able to determine the appropriate amount of Vitamin E TPGS for the first portion and second portion. Preferably, the second portion of the non-ionic surfactant is initially heated to a temperature ranging from about 50° C. to about 70° C.

The mixture is heated during granulation to a temperature below the melting temperature of the Vitamin E TPGS, which is about 38° C. to about 41° C. The temperature of the mixture itself is referred to as “product temperature”. Preferably, the product temperature is maintained below about 38° C. during granulation.

After cooling, the granules may be milled and subsequently screened through a sieve. Suitable particle sizes for the granules include but are not limited to the range between about 0.2 to about 3 mm, about 0.5 to about 2.5 mm, about 1 to about 2 mm, or about 1.25 to about 2 mm. Once the melt granulated granules are obtained, the granules may be formulated into a solid oral dosage form (e.g., tablets, pills, lozenges, caplets, capsules or sachets) or a liquid oral dosage form (e.g., drinks, solutions or beverages). Preferably, the granules are formulated into a capsule or sachet. The granules may be filled into a capsule or sachet manually. The granules may alternatively be filled into capsules using a Zinazi encapsulator.

The present invention further relates to a oral dosage form comprising the pharmaceutical composition of a therapeutic compound of formula (I), which comprises granules that comprise at least therapeutic compound of formula (I) (see below), particularly 2-Methyl-2-[4-(3-methyl-2-oxo-8-quinolin-3-yl-2,3-dihydro-imidazo[4,5-c]quinolin-1-yl)-phenyl]-propionitrile or 8-(6-methoxy-pyridin-3-yl)-3-methyl-1-(4-piperazin-1-yl-3-trifluoromethyl-phenyl)-1,3-dihydro-imidazo[4,5-c]quinolin-2-one, or a tautomer thereof, or a pharmaceutically acceptable salt, or a hydrate or solvate thereof; at least one non-ionic surfactant that is Vitamin E-TPGS in an amount ranging from about 15 to about 80% by weight of the composition; and at least one a dissolution enhancing agent selected from polyethylene glycol, polyethylene oxide, and any combination of the foregoing. The oral dosage forms useful for the present invention include both solid or liquid dosage forms. Examples of suitable solid oral dosage forms include but are not limited to tablets, pills, lozenges, caplets, capsules or sachets. Examples of suitable liquid dosage forms include but are not limited to drinks, solutions or beverages.

In the preferred embodiment, the oral dosage form is a solid oral dosage form. Preferably, the oral dosage form is a capsule or sachet. In accordance with the present invention, the pharmaceutical composition may be packaged in single or multiple dose sachets to be dissolved within the mouth, under the tongue, or are formulated in form of a tablet.

A tablet may be used as a suitable oral dosage form. After the granules are cooled and sieved, the mixture may be further blended, e.g. through a V-blender and subsequently compressed or molded into a monolithic tablet. The tablet can be optionally coated with a functional or non-functional coating as known in the art. Examples of coating techniques include, but are not limited to, sugar coating, film coating, microencapsulation and compression coating. Types of coating include, but are not limited to enteric coatings, sustained release coatings, and controlled release coating.

Any capsule as known in the art may be used to encapsulate the pharmaceutical composition of the present invention. An example of such capsule is hard gelatin capsules, for example CONI-SNAP manufactured by Capsugel of Morris Plains, N.J. Suitable sizes for such capsules include, but are not limited to size Nos. 00 through 5.

Any sachet as known in the art may be used as the dosage form for the pharmaceutical composition of the present invention. An example of such sachet is the MONUROL (fosfomycin tromethamine) sachet manufactured by Zambon Switzerland Ltd.

Any liquid or semi-liquid drink/food which can be safely consumed by the subject can be used to administer the pharmaceutical composition of the present invention. One of ordinary skill would understand how to calculate and administer the proper dosage of the compound of formula (I) in such liquid or semi-liquid drink/food. In such embodiment, the granules of the pharmaceutical composition may be mixed into a liquid drink (e.g., water, milk, or a soda) or a semi-liquid food (e.g., yogurt).

The oral dosage forms of the present invention may optionally further comprise additional conventional excipients used for pharmaceuticals. Examples of such excipients include, but are not limited to, disintegrants, binders, lubricants, glidants, stabilizers, and fillers, diluents, colorants, flavours and preservatives. One of ordinary skill in the art may select one or more of the aforementioned excipients with respect to the particular desired properties of the solid oral dosage form by routine experimentation and without any undue burden. The amount of each excipient used may vary within ranges conventional in the art. The following references which are all hereby incorporated by reference disclose techniques and excipients used to formulate oral dosage forms. See The Handbook of Pharmaceutical Excipients, 4^{th} edition, Rowe et al., Eds., American Pharmaceuticals Association (2003); and Remington: the Science and Practice of Pharmacy, 20^{th} edition, Gennaro, Ed., Lippincott Williams & Wilkins (2003).

These optional additional conventional excipients may be incorporated into the oral dosage form either by incorporating the one or more conventional excipients into the initial mixture before or during melt granulation or by combining the one or more conventional excipients with the granules in the oral dosage form. In the latter embodiment, the combined mixture may be further blended, e.g., through a V-blender, and subsequently compressed or molded into a tablet, for example a monolithic tablet, encapsulated by a capsule, or filled into a sachet.

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 5% by weight of composition.

Examples of pharmaceutically acceptable binders include, but are not limited to, starches; celluloses and derivatives thereof, for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be present in an amount from about 0% to about 50%, e.g., 2-20% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 80% by weight of the composition.

The utility of all the pharmaceutical compositions of the present invention may be observed in standard clinical test, for example, known indications of drug dosages giving therapeutically effective blood levels of the therapeutic compound; for example using dosages in the range from about 3 mg to approximately 5 g, from approximately 10 mg to approximately 1.5 g, preferably from approximately 100 mg to about 1600 mg per person, preferably from approximately 800 mg to about 1600 mg per person, preferably from about 800 mg to about 1400 mg per person, preferably from about 100 mg to about 1000 mg per person per day, or about 200 mg to about 400 mg per person per day, divided preferably into 1 to 3 single doses which may, for example, be of the same size.

The pharmaceutical compositions of the present invention are useful for the treatment of a proliferative disease, particularly a proliferative disease which responds to inhibition of lipid kinases and/or PI3kinase-related protein kinases, selected from a benign or malignant tumor, carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, an epidermal hyperproliferation, endometrial cancer, psoriasis, prostate hyperplasia, a neoplasia, a neoplasia of epithelial character, lymphomas, a mammary carcinoma or a leukemia. Other diseases include Cowden syndrome, Lhermitte-Dudos disease and Bannayan-Zonana syndrome, or diseases in which the PI3K/PKB pathway is aberrantly activated. Additional diseases which may be treated with the pharmaceutical composition of the present invention are disclosed in WO2006/122806, which is hereby incorporated by reference. Most preferably, the disease treated is a proliferative disease which response to inhibition of the PI3 kinase.

The pharmaceutical compositions of the present invention may further be useful for the treatment of those mTOR kinase dependent diseases disclosed in WO2008/103636, which is hereby incorporated by reference. Syndromes with an established or potential molecular link to dysregulation of mTOR kinase activity are, for instance, described in “K. Inoki et al.; Disregulation of the TSC-mTOR pathway in human disease, Nature Genetics, vol 37, 19-24”; “D. M. Sabatini; mTOR and cancer: insights into a complex relationship, Nature Reviews, vol. 6, 729-734”; and in “B. T. Hennessy et al.; Exploiting the PI3K/Akt pathway for cancer drug discovery, Nature Reviews, vol. 4, 988-1004”, which all are, including the referenced cited therein, hereby incorporated into the present application by reference, and are as follows: Organ or tissue transplant rejection, e.g. for the treatment of recipients of e.g. heart, lung, combined heart-lung, liver, kidney, pancreatic, skin or corneal transplants; graft-versus-host disease, such as following bone marrow transplantation; restenosis tuberous sclerosis; lymphangioleiomyomatosis; retinitis pigmentosis; autoimmune diseases including encephalomyelitis, insulin-dependent diabetes mellitus, lupus, dermatomyositis, arthritis and rheumatic diseases; steroid-resistant acute lymphoblastic leukaemia; fibrotic diseases including scleroderma, pulmonary fibrosis, renal fibrosis, cystic fibrosis; pulmonary hypertension; immunomodulation; multiple sclerosis; VHL syndrome; Carney complex; familial adenonamtous polyposis; juvenile polyposis syndrome; birt-Hogg-Duke syndrome; familial hypertrophic cardiomyopathy; Wolf-Parkinson-White syndrome; neurodegenerative disorders such as Parkinson's, Huntington's, Alzheimer's and dementias caused by tau mutations, spinocerebellar ataxia type 3, motor neuron disease caused by SOD1 mutations, neuronal ceroid lipofucinoses/Batten disease (pediatric neurodegeneration); wet and dry macular degeneration; muscle wasting (atrophy, cachexia) and myopathies such as Danon's disease; bacterial and viral infections including M. tuberculosis, group A streptococcus, HSV type I, HIV infection; neurofibromatosis including neurofibromatosis type 1; Peutz-Jeghers syndrome or further any combinations thereof.

The pharmaceutical compositions of the present invention may be administered in an amount sufficient to provide to provide a AUC of about 200 to about 70,000 ng*h/mL of the compound of formula (I), or a tautomer thereof, or a pharmaceutically acceptable salt, or a hydrate or solvate thereof, in the subject's plasma. In other such embodiments, the amount administered is sufficient to provide an AUC of about 500 to 43,000 ng*h/mL in the subjects plasma. In other such embodiments, the AUC is about 1,000 to 21,000 ng*h/mL in the subject's plasma.

The pharmaceutical compositions of the present inventions of the invention may be in a capsule or sachet sufficient to provide an AUC of about 200 to about 70,000 ng*h/mL of the compound of formula (I), or a tautomer thereof, or a pharmaceutically acceptable salt, or a hydrate or solvate thereof, in a subject's plasma after administration to the subject. In other such embodiments, the amount administered is sufficient to provide an AUC of about 500 to 43,000 ng*h/mL in the subjects plasma. In other such embodiments, the AUC is about 1,000 to 21,000 ng*h/mL in the subject's plasma.

The pharmaceutical compositions of the present inventions may also be in a sachet sufficient to provide at least an AUC of about 400 to about 15,000 ng*h/mL of the compound of formula (I), or a tautomer thereof, or a pharmaceutically acceptable salt, or a hydrate or solvate thereof, in the subject's plasma after administration to the subject.

The present invention further relates to a method of treatment of a subject suffering from a disease, condition or disorder treatable with a therapeutic compound of formula (I) comprising administering a therapeutically effective amount of a pharmaceutical composition of the present invention to a subject in need of such treatment. The diseases and pharmacokinetic attributes described above are further embodiments of this aspect of the present invention and incorporated herein by reference.

Additionally, the present invention relates to the use of a pharmaceutical composition of the present invention comprising a compound of formula (I), particularly COMPOUND A or COMPOUND B, in the manufacture of a medicament for the treatment of a proliferative disease selected from a benign or malignant tumor, carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, an epidermal hyperproliferation, endometrial cancer, psoriasis, prostate hyperplasia, a neoplasia, a neoplasia of epithelial character, lymphomas, a mammary carcinoma or a leukemia, Cowden syndrome, Lhermitte-Dudos disease, Bannayan-Zonana syndrome, or diseases in which the PI3K/PKB pathway is aberrantly activated. The present invention further relates to the use of a pharmaceutical composition of the present invention comprising a compound of formula (I), particularly COMPOUND A or COMPOUND B, in the manufacture of a medicament for the treatment of a mTOR kinase dependent disease as defined in WO2008/103636.

The present invention further relates to kit comprising (a) an oral pharmaceutical composition which comprises granules that comprise a therapeutic compound of formula (I), at least one non-ionic surfactant that is Vitamin E TPGS in an amount ranging from about 15 to 80% by weight of the composition and at least one dissolution enhancing agent selected from polyethylene glycol, polyethylene oxide, and any combination of the foregoing, and (b) written instructions, wherein such written instructions provide that such oral pharmaceutical composition may be taken between thirty minutes prior to the consumption of food until about two hours after the consumption of food. Preferably, said written instructions provide that such oral pharmaceutical composition may be taken immediately to about thirty minutes after the consumption of food.

The kit may comprise next to the written instructions as mentioned above about 3 mg to approximately 5 g, from approximately 10 mg to approximately 1.5 g, preferably from approximately 100 mg to about 1600 mg per person, preferably from approximately 800 mg to about 1600 mg per person, preferably from about 800 mg to about 1400 mg per person, preferably from about 100 mg to about 1000 mg per person per day, or about 200 mg to about 400 mg per person per day, divided preferably into 1 to 3 single doses which may, for example, be of the same size.

The present invention further relates to the use of the pharmaceutical composition of the present invention for the treatment of a proliferative disease or a mTOR kinase dependent disease comprising administering to a subject in need thereof a pharmaceutical composition which comprises granules that comprise a therapeutic compound of formula (I), at least one non-ionic surfactant that is Vitamin E TPGS in an amount ranging from about 15 to 80% by weight of the composition and at least one dissolution enhancing agent selected from polyethylene glycol, polyethylene oxide, and any combination of the foregoing, with food, wherein the administration of such pharmaceutical composition with food results in an increase in the bioavailability of the compound of formula (I) as compared to administration of the pharmaceutical composition to a subject without food.

In one embodiment, provided is the use of the pharmaceutical composition of the present invention for the treatment of a proliferative disease or a mTOR kinase dependent disease, wherein the pharmaceutical composition is administered to a subject in need thereof with food, wherein the Cₘₐₓ is increased as compared to administering the pharmaceutical composition without food. In a further embodiment, the Cₘₐₓ is increased by at least 20% as compared to administering the pharmaceutical composition without food.

In one embodiment, provided is the use of the pharmaceutical composition of the present invention for the treatment of a proliferative disease or a mTOR kinase dependent disease, wherein the pharmaceutical composition is administered to a subject in need thereof with food, wherein the AUCₗₐₛₜ is increased as compared to administering the pharmaceutical composition without food. In a further embodiment, the AUCₗₐₛₜ is increased by at least 15% as compared to administering the pharmaceutical composition without food.

The present invention further relates to the use of the pharmaceutical composition of the present invention for the treatment of a proliferative disease or a mTOR kinase dependent disease comprising administering to a subject in need thereof a pharmaceutical composition which comprises granules that comprise a therapeutic compound of formula (I), at least one non-ionic surfactant that is Vitamin E TPGS in an amount ranging from about 15 to 80% by weight of the composition and at least one dissolution enhancing agent selected from polyethylene glycol, polyethylene oxide, and any combination of the foregoing, with food, wherein the administration of such pharmaceutical composition with food results in an increase in the bioavailability of the compound of formula (I) as compared to administration of the compound of formula (I) to a subject without food.

In one embodiment, provided is the use of the pharmaceutical composition of the present invention for the treatment of a proliferative disease or a mTOR kinase dependent disease, wherein the pharmaceutical composition is administered to a subject in need thereof with food, wherein the Cₘₐₓ is increased as compared to administering the compound of formula (I) to a subject without food. In a further embodiment, the Cₘₐₓ is increased by at least 20% as compared to administering the compound of formula (I) without food.

In one embodiment, provided is the use of the pharmaceutical composition of the present invention for the treatment of a proliferative disease or a mTOR kinase dependent disease, wherein the pharmaceutical composition is administered to a subject in need thereof with food, wherein the AUCₗₐₛₜ is increased as compared to administering the compound of formula (I) to a subject without food. In a further embodiment, the AUCₗₐₛₜ is increased by at least 15% as compared to administering the compound of formula (I) without food.

The present invention further relates to a method of treatment of a proliferative disease or a mTOR kinase dependent disease comprising administering to a subject in need thereof a pharmaceutical composition of the present invention comprising a therapeutically effective amount of the compound of formula (I) with food, wherein the administration of such pharmaceutical composition with food results in an increase in the bioavailability of the compound of formula (I) as compared to administration of the pharmaceutical composition to a subject without food.

In one embodiment, provided is to a method of treatment of a proliferative disease or a mTOR kinase dependent disease comprising administering to a subject in need thereof a pharmaceutical composition of the present invention comprising a therapeutically effective amount of the compound of formula (I) with food, wherein the AUCₗₐₛₜ is increased as compared to administering the pharmaceutical composition without food. In a further embodiment, the AUCₗₐₛₜ is increased by at least 15% as compared to administering the pharmaceutical composition without food.

In one embodiment, provided is to a method of treatment of a proliferative disease or a mTOR kinase dependent disease comprising administering to a subject in need thereof a pharmaceutical composition of the present invention comprising a therapeutically effective amount of the pharmaceutical composition with food, wherein the Cₘₐₓ is increased as compared to administering the pharmaceutical composition without food. In a further embodiment, the Cₘₐₓ is increased by at least 20% as compared to administering the pharmaceutical composition without food.

The present invention further relates to a method of treatment of a proliferative disease or a mTOR kinase dependent disease comprising administering to a subject in need thereof a pharmaceutical composition of the present invention comprising a therapeutically effective amount of the compound of formula (I) with food, wherein the administration of such pharmaceutical composition with food results in an increase in the bioavailability of the compound of formula (I) as compared to administration of the compound of formula (I) to a subject without food.

In one embodiment, provided is to a method of treatment of a proliferative disease or a mTOR kinase dependent disease comprising administering to a subject in need thereof a pharmaceutical composition of the present invention comprising a therapeutically effective amount of the compound of formula (I) with food, wherein the AUCₗₐₛₜ is increased as compared to administering the pharmaceutical composition without food. In a further embodiment, the AUCₗₐₛₜ is increased by at least 15% as compared to administering the compound of formula (I) to a subject without food.

In one embodiment, provided is to a method of treatment of a proliferative disease or a mTOR kinase dependent disease comprising administering to a subject in need thereof a pharmaceutical composition of the present invention comprising a therapeutically effective amount of the pharmaceutical composition with food, wherein the Cₘₐₓ is increased as compared to administering the pharmaceutical composition without food. In a further embodiment, the Cₘₐₓ is increased by at least 20% as compared to administering the compound of formula (I) to a subject without food.

The following examples are illustrative, but do not serve to limit the scope of the invention described herein. The examples are meant only to suggest a method of practicing the present invention.

Quantities of ingredients, represented by percentage by weight of the pharmaceutical composition, used in each example are set forth below. For a capsule or sachet, when calculating the weight of the pharmaceutical composition (i.e., the capsule or sachet fill weight), the weight of the capsule shell or sachet itself is excluded from the calculation.

### Example 1

| | | Amount per | Amount per |
|---|---|---|---|
| | | dosage unit | dosage unit |
| | | (mg) - | (mg) - |
| | Percentage | 200 mg free | 400 mg free |
| Ingredient | (w/w) | base | base |
| 2-methyl-2-[4-(3- | 39.1% | 273.4 | 546.8 |
| methyl-2-oxo-8- | | | |
| quinolin-3-yl-2,3- | | | |
| dihydro-imidazo[4,5- | | | |
| c]quinolin-1-yl)-phenyl]- | | | |
| propionitrile | | | |
| monotosylate salt | | | |
| D-alpha-tocopheryl | 41.1% | 288.0 | 576.0 |
| polyethylene glycol | | | |
| 1000 succinate | | | |
| Mannitol | 11.2% | 78.6 | 157.2 |
| Polyethylene glycol | 4.0% | 28.0 | 56.0 |
| (PEG) 3350 | | | |
| Crospovidone | 4.57% | 32.0 | 64.0 |
| TOTAL FILL WEIGHT | 100.0% | 700.0 | 1400.0 |

Each of the above ingredients are individually measured and weighed. The Vitamin E TPGS is commercially available from the company Eastman Fine Chemicals Kingsport, Tex., USA. The mannitol is commercially available from the company Roquette GMBH. The PEG3350 is commercially available from the company Clariant GMBH. The crospovidone is commercially available from the company International Specialty Products Corporation. Subsequently, the total amount of Vitamin E TPGS is divided into two portions having 56% (part 1) and 44% (part 2) of the total amount. Part 1 of the Vitamin E TPGS is loaded into a suitable sized vessel and frozen overnight or by dry ice, and then the frozen Vitamin E TPGS is dry milled at medium speed and sieved through a Fitzmill homoloid machine JT fitted with a Screen #0079.

For the granulation process, each of the following ingredients are loaded into a suitable size granulator bowl in a Collette™ GRAL™ high-sheer mixer: (1) 2-methyl-2-[4-(3-methyl-2-oxo-8-quinolin-3-yl-2,3-dihydro-imidazo[4,5-c]quinolin-1-yl)-phenyl]-propionitrile monotosylate salt, (2) Mannitol, (3) Crospovidone, (4) PEG 3350, and (5) Vitamin E PEG Succiniate (Part I). The materials are dry mixed for 5 minutes with the PLOW set at Low Speed and record the mixed powder temperature. The mixture is then allowed to cool in the granulator until a temperature less than or equal to 22° C. is obtained.

Part 2 of the Vitamin E TPGS is loaded into a suitable size vessel and heated until melting to a temperature range of 50° C. to 70° C.

After starting the Collette™ GRAL™ high-sheer mixer with the PLOW set at Low Speed, the required amount of Part 2 of the Vitamin E TPGS is added to the mixture within two (2) minutes. The subsequent mixture is allowed to continue mixing in the high sheer mixer (with PLOW and Chopper set at High Speed) for an additional one (1) minute. After setting the PLOW and CHOPPER to Low Speed, the mixture is allowed to continue mixing in the high sheer granulator for a maximum time of 25 minutes until proper granules are formed. The product temperature should be maintained less than 38° C. throughout mixing. The formed granules are manually sieved using a size 14 mesh screen and placed into a suitable container.

After the sieving is completed, all granules are cooled using either a freezer or dry ice to a temperature of 0° C. or below. The cooled granules are further sieved using an Frewitt GLA oscillator fitted with a size 14 mesh screen to reduce or remove oversized granules.

The sieved granules obtained from the foregoing process are loaded into a suitable size bin tote of a bin blender and blended together for 150 revolutions. After blending, the granules are manually filled into sachets.

### Example 2

| | Percentage | Amount per dosage unit (mg) - |
|---|---|---|
| Ingredient | (w/w) | 200 mg free base |
| 2-methyl-2-[4-(3- | 39.1% | 273.4 |
| methyl-2-oxo-8- | | |
| quinolin-3-yl-2,3- | | |
| dihydro-imidazo[4,5- | | |
| c]quinolin-1-yl)- | | |
| phenyl]-propionitrile | | |
| monotosylate salt | | |
| D-alpha-tocopheryl | 41.1% | 288.0 |
| polyethylene glycol | | |
| 1000 succinate | | |
| Mannitol | 11.2% | 78.6 |
| Polyethylene glycol | 4.6% | 32 |
| 3350 | | |
| Crospovidone | 4.0% | 28 |
| TOTAL | 100.0% | 700.0 |

Example 2 is made using the same method as disclosed for Example 1; however, the final granules are filled into capsules by using the Zanasi encapsulation machine.

### Example 3

| | | | Amount |
|---|---|---|---|
| | | Amount per | per dosage |
| | Percentage | dosage unit (mg) - | unit (mg) - 50 mg |
| Ingredient | (w/w) | 200 mg free base | free base |
| 2-methyl-2-[4-(3- | 34.2% | 273.4 | 68.4 |
| methyl-2-oxo-8- | | | |
| quinolin-3-yl-2,3- | | | |
| dihydro- | | | |
| imidazo[4,5- | | | |
| c]quinolin-1-yl)- | | | |
| phenyl]-propionitrile | | | |
| monotosylate salt | | | |
| D-alpha-tocopheryl | 36.0% | 288.0 | 72.00 |
| polyethylene glycol | | | |
| 1000 succinate | | | |
| Mannitol | 21.3% | 170.6 | 42.7 |
| Polyethylene glycol | 4.5% | 36.0 | 9.00 |
| 3350 | | | |
| Crospovidone | 32.0% | 32 | 8.00 |
| TOTAL | 100.0% | 800.0 | 200.0 |

Example 3 is made using the same method as disclosed for Example 1; however, the total amount of D-alpha-tocopheryl polyethylene glycol 1000 succinate is added to the initial mixture and the granules are formed by melt extrusion using a 17 mm melt extruder, subsequently sieved using a Fitz Homoloid Mill fitted with a Screen No. 0079, and finally manually filled into capsules. 273.4 mg and 68.4 mg of 2-methyl-2-[4-(3-methyl-2-oxo-8-quinolin-3-yl-2,3-dihydro-imidazo[4,5-c]quinolin-1-yl)-phenyl]-propionitrile monotosylate salt is equivalent to 200 mg and 50 mg of free base respectively.

### Example 4

| | | | Amount | | Amount |
|---|---|---|---|---|---|
| | | Amount | per | Amount | per |
| | | per | dosage | per | dosage |
| | Percentage | dosage | unit | dosage | unit |
| Ingredient | (w/w) | unit (mg) | (mg) | unit (mg) | (mg) |
| 2-methyl-2-[4- | 59.4% | 136.7 | 205.1 | 273.4 | 341.8 |
| (3-methyl-2-oxo- | | | | | |
| 8-quinolin-3-yl- | | | | | |
| 2,3-dihydro- | | | | | |
| imidazo[4,5- | | | | | |
| c]quinolin-1-yl)- | | | | | |
| phenyl]- | | | | | |
| propionitrile | | | | | |
| monotosylate | | | | | |
| salt | | | | | |
| D-alpha- | 33.6% | 77.3 | 116.0 | 154.6 | 193.3 |
| tocopheryl | | | | | |
| polyethylene | | | | | |
| glycol 1000 | | | | | |
| succinate | | | | | |
| Polyethylene | 7.0% | 16.0 | 24.0 | 32.0 | 40.0 |
| glycol 3350 | | | | | |
| TOTAL | 100% | 100% | 100% | 100% | 100% |

Example 4 is made using the same process as Example 1; however no mannitol or crospovidone is added to the initial mixture.

### Example 5

### Dog Studies

The following two formulation variants are prepared in situ prior to administration to dogs:

| Ingredients | Variant 1 (mg/unit) | | Variant 2 (mg/unit) | |
|---|---|---|---|---|
| 2-methyl-2-[4-(3-methyl-2- | 68.5 | mg* (10.5%) | 68.5 | mg* (38.5%) |
| oxo-8-quinolin-3-yl-2,3- | | | | |
| dihydro-imidazo[4,5- | | | | |
| c]quinolin-1-yl)-phenyl]- | | | | |
| propionitrile monotosylate | | | | |
| salt | | | | |
| D-alpha-tocopheryl | 500.0 | mg (77.0%) | 72.5 | mg (40.7%) |
| polyethylene glycol 1000 | | | | |
| succinate | | | | |
| Mannitol | 26.5 | mg (4.1%) | 23.0 | mg (12.9%) |
| Polyethylene glycol 3350 | 32.5 | mg (5%) | 8.0 | mg (4.5%) |
| Crospovidone | 22.5 | (3.5%) | 6.0 | mg (3.4%) |
| Fill weight/capsule | 650 | | 178 | |

*68.5 mg of 2-methyl-2-[4-(3-methyl-2-oxo-8-quinolin-3-yl-2,3-dihydro-imidazo[4,5-c]quinolin-1-yl)-phenyl]-propionitrile monotosylate salt is equivalent to 50 mg of the free base of 2-methyl-2-[4-(3-methyl-2-oxo-8-quinolin-3-yl-2,3-dihydro-imidazo[4,5-c]quinolin-1-yl)-phenyl]-propionitrile.

Both formulations are made according to the same process as Example 1; however, the total amount of D-alpha-tocopheryl polyethylene glycol 1000 succinate is added to the initial mixture and the granules are formed by melt extrusion using a 17 mm melt extruder and then subsequently sieved using a Fitz Homoloid Mill fitted with a Screen No. 0079.

### 2-Step Dissolution Test

Capsules containing either Compound A alone, Formulation Variants 1 or 2 are separately evaluated using a 2-Step Dissolution Test. This analysis compared the dissolution profile of capsules containing 68.5 mg per unit of 2-methyl-2-[4-(3-methyl-2-oxo-8-quinolin-3-yl-2,3-dihydro-imidazo[4,5-c]quinolin-1-yl)-phenyl]-propionitrile monotosylate salt (which is equivalent to 50 mg of the free base of 2-methyl-2-[4-(3-methyl-2-oxo-8-quinolin-3-yl-2,3-dihydro-imidazo[4,5-c]quinolin-1-yl)-phenyl]-propionitrile) at pH 2 and pH 6.8.

For this analysis, capsules containing either Compound A alone, Formulation Variant 1 or Variant 2 are placed in 900 mL of pH 2 HCl and then stirred in a USP Apparatus II rotating at 50 rpm for 10-100 minutes (with fast stir from about minute 60 to minute 90). At approximately the 100 minute time point, this medium is adjusted to pH 6.8 and to a volume of 1000 ml and then stirred in a USP Apparatus II rotating at 50 rpm. Samples are filtered using a 100 μm Varian Full Flow filter and analyzed by HPLC.

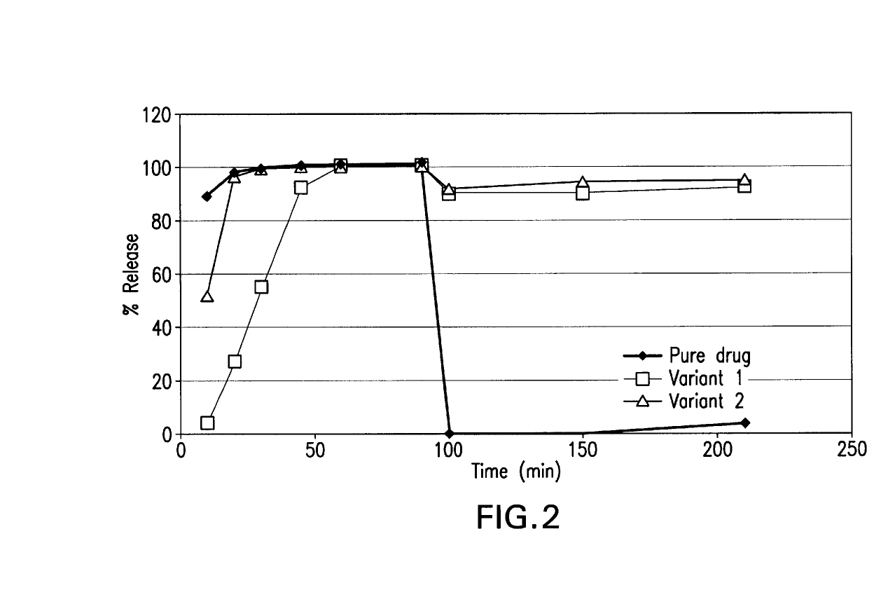

As shown in FIG. 2, the results of this two-Step Dissolution Test clearly demonstrated that the pharmaceutical composition of the present invention significantly improves the dissolution of Compound A as compared to Compound alone. In addition, the pharmaceutical composition of the present invention has a similar drug release at both pH 2 and pH 6.8 whereas Compound A alone has approximately 0% drug release at pH 6.8. This data further confirms that the pharmaceutical composition successfully maintains Compound A in its supersaturated concentrations.

### Dog Study

In this study, two groups (n=3 per group) of male pure-bread beagle dogs (originating from the ADME colony that originated from Marshall farms, North Rose, N.Y.) are orally administered two capsules containing an amount of 2-methyl-2-[4-(3-methyl-2-oxo-8-quinolin-3-yl-2,3-dihydro-imidazo[4,5-c]quinolin-1-yl)-phenyl]-propionitrile monotosylate salt equivalent to a total of 50 mg of the free base (referred to as “50 mg Compound A” for this study description).

Each dog weighed between 8 and 13 kg. During the study, the dogs are housed individually in properly marked cages and were identified by consecutive numbers. The dogs are weighted in the week before dosing.

For administration, each dog in Group 1 and 2 receives two capsules containing 25 mg Compound A followed by 50 mL of water via oral gavage. Group 1 is orally administered two 25 mg capsules having the equivalent to Formulation Variant 1, whereas Group 2 is orally administered two 25 mg capsules having the equivalent to Formulation Variant 2.

Serial blood samples are collected from each dog pre-dose and at 0.25, 0.5, 1, 2, 3, 4, 6, 8 and 24 hours after administration. Approximately 2 mL of venous whole blood is collected. Blood samples are collected via the cephalic vein into a 2.5 mL blood collection tube using EDTA as an anticoagulant. The samples are centrifuged and the plasma is separated and transferred to polypropylene cryovials. The plasma samples are stored at less than or equal to −20° C. prior to analysis.

The concentration of Compound A in the plasma is measured by a validated liquid chromatography-tandem mass spectrometry assay (LC-MS/MS) (Bioanalytics located in East Hanover, N.J.). The following instruments and settings are used for the LC-MS/MS:
Instrument: TomTec Quadra 96
LC condition: Column: ACE Phenyl, 3 μm, (50×4.6 mm), Mac-Mod Analytical); temperature: 30° C.; isocratic eluton: [A: 0.1% formic acid in water, B: 0.1% formic acid in acetonitrile:methanol (50:50, v/v), (A:B, 30:70, v/v)]; flow rate: 1.0 mL/min; injection volume: 10 μL.
LC system: Pumps: Shimadzu LC-20AD; Autosampler: Simadzu SIL 20-AC; Oven: Shimadzu CTO 20-AC.
Mass spectrometer: API4000, Applied Biosystems

Mass spectrometer settings: MS conditions (ESI: Source temperature: 450° C., voltage: 5500 V, Dwell time: 150 ms for Compound A and [m+4]Compound A.); Masses Compound A (Precursor ion: m/z 470.3; product ion: m/z 443.1; collision energy (CE): 49 (eV); declustering potential (DP): 146 (eV)); Masses ISTD (Precursor ion: m/z 474.3; product ion: m/z 447.3; collision energy (CE): 55 (eV); declustering potential (DP): 141 (eV)).

Concentration-time curves are analyzed using non-compartmental methods with the WinNonlin version 5.2 program (Pharsight Corporation, Mountain View, Calif.). The highest plasma parent compound concentration (Cmax) and times that occur (Tmax) are recorded. The AUCₗₐₛₜ is calculated using the linear trapezoidal rule.

The following results are obtained from the above dog study:

| Formulation | | | | AUCₗₐₛₜ | T_{1/2} |
|---|---|---|---|---|---|
| Variant | Dog | Tmax (hr) | Cmax (ng/ml) | (hr * ng/ml) | (hr) |
| 1 | Dog 1 | 2 | 78 | 436 | 5 |
| 1 | Dog 2 | 2 | 295 | 1850 | 5.7 |
| 1 | Dog 3 | 2 | 183 | 880 | 4.5 |
| Mean | | 2 | 185 | 1060 | 5.1 |
| CV % | | | 59 | 68 | |
| 2 | Dog 1 | 4 | 78.6 | 503 | 6.9 |
| 2 | Dog 2 | 2 | 23.1 | 225 | 7 |
| 2 | Dog 3 | 2 | 247 | 1270 | 6.4 |
| Mean | | 2.7 | 116 | 666 | 6.8 |
| CV % | | | 101 | 81 | |

The results of this dog study clearly shows that the pharmaceutical composition of the present invention significantly improves absorption and bioavailability of the therapeutic compound in vivo.

### Example 6

Healthy female human volunteers aged 18 to 55 years who are either post-menopausal or surgically sterile are orally administered a dose of 2-methyl-2-[4-(3-methyl-2-oxo-8-quinolin-3-yl-2,3-dihydro-imidazo[4,5-c]quinolin-1-yl)-phenyl]-propionitrile monotosylate salt equivalent to a total of 25 mg of the free base. Patients are orally administered a dose of 2-methyl-2-[4-(3-methyl-2-oxo-8-quinolin-3-yl-2,3-dihydro-imidazo[4,5-c]quinolin-1-yl)-phenyl]-propionitrile monotosylate salt equivalent to a total of 25 mg of the free base as either a Hard Gelatin Capsule standard formulation or Hard Gelatin Capsule comprising the pharmaceutical composition of the present invention (“Hard Gelatin Capsule SDS”).

The Hard Gelatin Capsule standard formulation is a wet-granulated pharmaceutical composition including an amount of 2-methyl-2-[4-(3-methyl-2-oxo-8-quinolin-3-yl-2,3-dihydro-imidazo[4,5-c]quinolin-1-yl)-phenyl]-propionitrile monotosylate salt equivalent to a total of 25 mg of the free base and corresponding to the following composition (wherein the amounts are percentages of the capsule fill weight):

| Component | Composition per unit (mg) (% per unit) | |
|---|---|---|
| INNER PHASE | | |
| 2-methyl-2-[4-(3-methyl-2- | 136.7 mg | (59.9%) |
| oxo-8-quinolin-3-yl-2,3- | | |
| dihydro-imidazo[4,5- | | |
| c]quinolin-1-yl)-phenyl]- | | |
| propionitrile monotosylate | | |
| salt | | |
| Lactose 200 Mesch | 41.0 mg | (18%) |
| Polyvinylpyrrolidone K30 | 7.8 mg | (3.42%) |
| Crospovidone XL | 9.8 mg | (4.27%) |
| OUTTER PHASE | | |
| Starch 1500 | 29.7 mg | (13.0%) |
| Lactose 200 mesh | — | |
| Aerosil | 1.14 | (0.50%) |
| Magnesium Stearate | 2.28 mg | (1.0%) |

136.7 mg of 2-methyl-2-[4-(3-methyl-2-oxo-8-quinolin-3-yl-2,3-dihydro-imidazo[4,5-c]quinolin-1-yl)-phenyl]-propionitrile monotosylate salt is equivalent to 100 mg of the free base of 2-methyl-2-[4-(3-methyl-2-oxo-8-quinolin-3-yl-2,3-dihydro-imidazo[4,5-c]quinolin-1-yl)-phenyl]-propionitrile. The Hard Gelatin Capsule SDS includes an amount of 2-methyl-2-[4-(3-methyl-2-oxo-8-quinolin-3-yl-2,3-dihydro-imidazo[4,5-c]quinolin-1-yl)-phenyl]-propionitrile monotosylate salt equivalent to a total of 25 mg of the free base and corresponds to the formulation disclosed in Example 3 herein.

The study is conducted in two parts involving: (a) Three treatment periods and six sequences (n=18) to test the effect of a light meal on drug exposure when patients are administered the Hard Gelatin Capsule standard formulation, and (b) Five Treatment periods and ten treatment sequences (n=20) to test the effect of drug formulation and the presence of a light meal as comparing the Hard Gelatin Capsule standard formulation with the Hard Gelatin Capsule SDS.

Each healthy subject is provided with a screening period of up to 2 weeks, a baseline period starting on the day prior to each dose, an in-house dosing and sample collection period of at least 24 hours after each dose, a washout period of at least 7 days after each dose, and an end of study visit. Healthy subjects are fasted overnight prior to receiving 25 mg (˜0.3 mg/kg) of either the Hard Gelatin Capsule standard formulation or Hard Gelatin Capsule SDS on an empty stomach followed by a further fast of 4 hours before a standard lunch (i.e., fasted) or 30 minutes after a light meal (i.e., fed). Blood is collected at selected time points over a 24 hour period following each dose. Samples are collected from healthy subjects under the fasted and fed conditions. For the Hard Gelatin Capsule standard formulation under fasting conditions, drug exposure is quantifiable up to 10 hours post-dose, whereas the other conditions tested are quantifiable up to 12 hours post-dose.

Venous whole blood samples are centrifuged and the serum or plasma is isolated. These serum or plasma samples is stored at ≦−15° C. prior to shipping for analysis by a validated liquid chromatography-tandem mass spectrometry assay (LC-MS/MS) (Bioanalytics, East Hanover, N.J. Values below the lower limit of quantification of approximately 1 ng/mL are reported as 0.0 ng/mL. The lower limit of quantification is approximately 1 ng/ml.

Concentration-time curves are analysed using non-compartmental methods (Pharsight WinNonlin version 5.2). AUC₀₋ₜₗₐₛₜ is the area under the concentration-time curve up to the last measurable concentration. AUC_{0-inf} is the area under the concentration-time curve extrapolated to infinity. Cₘₐₓ is the maximum (peak) concentration after oral drug administration. Tₗₐₛₜ is the time to the last measurable concentration. Tₘₐₓ is the time to reach maximum (peak) concentration following oral drug administration.

The following results are obtained from the above study:

| | Hard Gelatin | Hard Gelatin | Hard | Hard |
|---|---|---|---|---|
| | Capsule | Capsule | Gelatin | Gelatin |
| | standard | standard | Capsule | Capsule |
| | formulation | formulation | SDS | SDS |
| PK parameters | (Fasted) | (Fed) | (Fasted) | (Fed) |
| No. of healthy | 19 | 12 | 19 | 18 |
| subjects | | | | |
| Tₘₐₓ (h) | 1.5 | 3 | 1 | 2 |
| Cₘₐₓ (ng/ml) | 13.8434 | 14.5982 | 13.9639 | 18.2628 |
| AUC₀₋ₜₗₐₛₜ | 36.9194 | 42.5564 | 34.1146 | 51.8920 |
| (ng * hr/ml) | | | | |
| AUC_{0-inf} (ng * hr/ml) | 39.9523 | 45.8383 | 37.8396 | 55.1631 |
| Tₗₐₛₜ (h) | | | | |
| AUC_{0-inf} (CV (%) | 68.57 | 50.79 | 45.94 | 39.91 |
| Geometric mean) | | | | |
| Cₘₐₓ (CV (%) | 58.47 | 47.40 | 49.41 | 56.70 |
| Geometric mean) | | | | |

Under fasting conditions the SDS capsule formulation yields a mean total drug exposure (AUC_{0-inf}) and a mean maximum drug exposure (Cₘₐₓ) similar to the HG capsule formulation. The geometric mean ratio as well as the 90% CI of the Hard Gelatin Capsule SDS is within the no-effect boundaries (0.8-1.25), therefore under fasting conditions the Hard Gelatin Capsule standard formulation and Hard Gelatin Capsule SDS are biocomparable.

However, under fasting conditions the variability in total mean drug exposure for the Hard Gelatin Capsule SDS is less than the variability seen for the Hard Gelatin Capsule standard formulation (CV AUC_{0-inf} of 46% versus 68%), suggesting a positive effect on drug exposure by the Hard Gelatin Capsule SDS. For the Hard Gelatin Capsule standard formulation in the presence of a light meal the variability in total mean drug exposure was 51%, suggesting that the presence of food improves the consistency of drug absorption of the Hard Gelatin Capsule standard formulation. The variability in total mean drug exposure for the Hard Gelatin Capsule SDS is also improved in the presence of a light meal (CV AUC_{0-inf} of 46% versus 40%).

When given in the presence of a light meal the Hard Gelatin Capsule SDS improves both total and maximum mean drug exposure compared to the Hard Gelatin Capsule standard formulation (under both fasting and fed conditions). Based on the geometric mean ratios, the total mean drug exposure using the SDS capsule formulation in the presence of a light meal improves by 40% (90% Confidence Interval 1.16-1.70) and the maximum mean drug exposure improves by 31% (90% Confidence Interval 1.03-1.67) compared to the Hard Gelatin Capsule standard formulation under fasting conditions. Moreover, the mean total drug exposure improves from 45.8 ng·h/ml with the Hard Gelatin Capsule standard formulation to 55.2 ng*h/ml with the Hard Gelatin Capsule SDS in the presence of a light meal, an increase of 20%, suggesting that the increase in drug exposure is not solely due to the effect of food. The maximum mean drug exposure (Cₘₐₓ) improves from 14.6 ng/ml with the Hard Gelatin Capsule standard formulation to 18.3 ng/ml with the Hard Gelatin Capsule SDS in the presence of a light meal, an increase of 25% further confirming that the increase in drug exposure is not solely due to the effect of food.

In addition, the SDS capsule formulation in the presence of a light meal improves the bioavailability (including AUC_{0-inf}, AUCₗₐₛₜ, and Cₘₐₓ) as compared to the Hard Gelatin capsule SDS in the fasted state.

### Example 7

Patients having metastatic/advanced solid tumors are orally administered with either a hard gelatin capsule comprising a pharmaceutical composition of the present invention (referred to as a “special delivery system capsule” or “SDS capsule” for this study description), or a sachet comprising a pharmaceutical composition of the present invention (referred to as a “special delivery system sachet or “SDS sachet” for this study description). Each patient is administered a suitable amount of 2-methyl-2-[4-(3-methyl-2-oxo-8-quinolin-3-yl-2,3-dihydro-imidazo[4,5-c]quinolin-1-yl)-phenyl]-propionitrile monotosylate salt.

In the study, eleven (11) patients are treated with SDS capsules containing a total amount of 2-methyl-2-[4-(3-methyl-2-oxo-8-quinolin-3-yl-2,3-dihydro-imidazo[4,5-c]quinolin-1-yl)-phenyl]-propionitrile monotosylate salt equivalent to 1000 mg free base, six (6) patients are treated with SDS capsules containing a total amount of 2-methyl-2-[4-(3-methyl-2-oxo-8-quinolin-3-yl-2,3-dihydro-imidazo[4,5-c]quinolin-1-yl)-phenyl]-propionitrile monotosylate salt equivalent to 800 mg free base, and five (5) patients are treated with the SDS capsule containing a total amount of 2-methyl-2-[4-(3-methyl-2-oxo-8-quinolin-3-yl-2,3-dihydro-imidazo[4,5-c]quinolin-1-yl)-phenyl]-propionitrile monotosylate salt equivalent to 400 mg free base. The SDS capsule used in the study is available in doses equivalent to 200 mg free base and corresponds to the formulation disclosed in Example 3 herein. The SDS capsule is administered orally once per day with food on a continuous daily dosing schedule.

Six (6) patients are treated with the SDS sachets containing a total amount of 2-methyl-2-[4-(3-methyl-2-oxo-8-quinolin-3-yl-2,3-dihydro-imidazo[4,5-c]quinolin-1-yl)-phenyl]-propionitrile monotosylate salt equivalent to 800 mg free base, four (4) patients are treated with the SDS sachets containing a total amount of 2-methyl-2-[4-(3-methyl-2-oxo-8-quinolin-3-yl-2,3-dihydro-imidazo[4,5-c]quinolin-1-yl)-phenyl]-propionitrile monotosylate salt equivalent to 1000 mg free base, nine (9) patients are treated with the SDS sachets containing a total amount of 2-methyl-2-[4-(3-methyl-2-oxo-8-quinolin-3-yl-2,3-dihydro-imidazo[4,5-c]quinolin-1-yl)-phenyl]-propionitrile monotosylate salt equivalent to 1400 mg free base, and seven (7) patients are treated with the SDS sachets containing a total amount of 2-methyl-2-[4-(3-methyl-2-oxo-8-quinolin-3-yl-2,3-dihydro-imidazo[4,5-c]quinolin-1-yl)-phenyl]-propionitrile monotosylate salt equivalent to 1600 mg free base. The 800 mg SDS sachet used in the study is available in doses equivalent to 400 mg free base and having the composition of the 400 mg formulation described Example 1 herein. The 1000 mg, 1200 mg and 1600 mg SDS sachets used in the study have the composition of the formulations of the present invention. To administer the SDS sachet, patients are instructed to suspend the SDS sachets in a glass of fresh water (approximately 1 dL), then to drink the content, then to rinse the glass with approximately 0.25 dL of water and then to drink the content while rinsing the mouth before swallowing. The SDS sachet is administered orally once per day with food on a continuous daily dosing schedule.

Blood samples are collected from each patient of the study. All blood samples are taken by either direct venipuncture or an indwelling cannula inserted into the forearm vein. At specified time points, 2 mL of blood is collected in tubes containing EDTA. The tubes are kept in an ice water bath at approximately 4° C. for ≦60 minutes during the sampling period. The tubes are centrifuged at between 3° C. and 5° C. for 10 minutes at approximately 100 g to separate plasma. Immediately after centrifugation, 1 mL plasma is transferred to a 2-mL polypropylene screw-cap tube that is kept on dry ice. The tubes are placed in a freezer set at <−15° C. until analysis.

The obtained plasma samples are assayed for drug concentrations using a validated liquid chromatography-tandem mass spectrometry assay (LC-MS/MS). Values below the lower limit of quantification of approximately 1 ng/mL are reported as 0.0 ng/mL.

The following results are obtained from the above study:

TABLE 1
Cₘₐₓ are reported as Median [Min-Max] (N)

| Dose (qd) | Formulation | Day 1 | Day 8 | Day 28 |
|---|---|---|---|---|
| 400 mg | SDS Capsule | 131 [53.8-405] (5) | 982 [107-1450] (5) | 700 [192-1820] (5) |
| 800 mg | SDS Capsule | 164 [36.5-1290] (6) | 1115 [362-3960] (6) | 1360 [207-3890] (5) |
| | SDS Sachet | 191 [38.2-656] (6) | 945 [116-1360] (5) | 668 [410-705] (3) |
| 1000 mg | SDS Capsule | 309 [25.2-771] (11) | 1090 [142-3230] (11) | 701 [76.4-4100] (6) |
| | SDS Sachet | 341 [136-1150] (4) | 996 [353-2390] (4) | 1572 [943-2200] (2) |
| 1400 mg | SDS Sachet | 385 [125-2720] (9) | 1181 [224-3220] (6) | 891 [367-3330] (6) |
| 1600 mg | SDS Sachet | 251 [163-599] (7) | 1945 [434-3220] (6) | 1590 [971-3330] (5) |

TABLE 2
AUC₀₂₄ are reported as Median [Min-Max] (N)

| Dose (qd) | Formulation | Day 1 | Day 8 | Day 28 |
|---|---|---|---|---|
| 400 mg | SDS Capsule | 1581 [517-3527] (5) | 10281 [1113-14302] (4) | 6657 [699.55-30859] (5) |
| 800 mg | SDS Capsule | 2886 [220-17276 ] (6) | 19770 [4872-50499](4) | 20888 [1666-50112] (5) |
| | SDS Sachet | 1003 [499-10600] (6) | 13055 [767-21346] (5) | 5896.3 [2978-13674] (3) |
| 1000 mg | SDS Capsule | 2956 [198-12445] (11) | 12195 [1185-67450] (11) | 6583 [592-74070] (5) |
| | SDS Sachet | 4149 [1395-18133] (4) | 13336.05 [4737-37608] (4) | 23299 [12965-33634] (2) |
| 1400 mg | SDS Sachet | 5118 [1335-27088 ] (9) | 6353 [162-56400] (6) | 18019 [16566-67455] (5) |
| 1600 mg | SDS Sachet | 2553 [1244-6860] (7) | 21777 [5504-37989] (7) | 14434 [7346-33221] (5) |

At the dose of 800 mg, the tmax with the SDS sachet is slightly shorter compared to the SDS capsule with median values between 1.5 and 4 hrs but it increases to 4 to 6 hrs at the 1400 mg and 1600 mg doses. The accumulation is variable from patient to patient. The accumulation ratio remains moderate for the 800 and 1400 mg doses, 3.7 and 2.7 (median) but increased to 6.9 at 1600 mg. Despite this accumulation, the median t_{1/2} is estimated between 4 and 8 hrs without major difference between doses and visits.

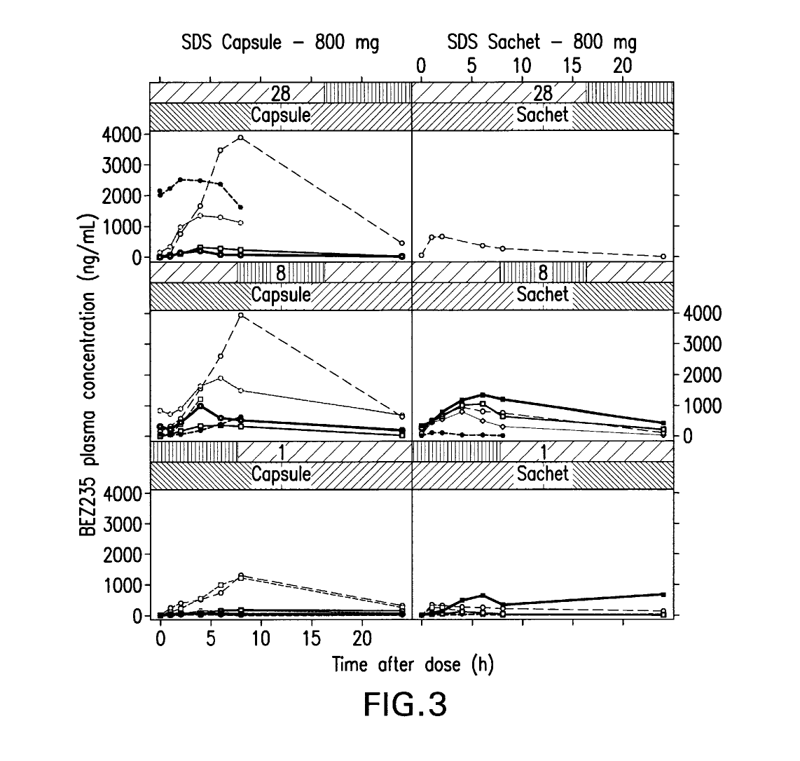

As shown by the results set forth in Tables 1 and 2, both the SDS capsule and SDS sachet formulations improve the exposure to the therapeutic compound indicating that the SDS composition improves dissolution and absorption of the compound. Surprisingly, those patients treated with the SDS sachet formulation display more consistency in their pharmacokinetic profile than seen for the SDS capsule formulation. The variability in total mean drug exposure for the SDS sachet is less than the variability seen for the SDS capsule. (See FIG. 3, which is based upon the preliminary data)

1. A pharmaceutical composition comprising granules that comprise:
(a) a therapeutic compound of formula (I) 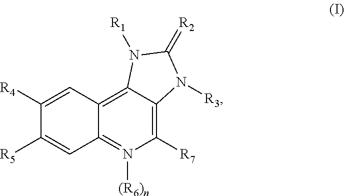
wherein
R₁ is naphthyl or phenyl wherein said phenyl is substituted by one or two substituents independently selected from the group consisting of Halogen; lower alkyl unsubstituted or substituted by halogen, cyano, imidazolyl or triazolyl; cycloalkyl; amino substituted by one or two substituents independently selected from the group consisting of lower alkyl, lower alkyl sulfonyl, lower alkoxy and lower alkoxy lower alkylamino; piperazinyl unsubstituted or substituted by one or two substituents independently selected from the group consisting of lower alkyl and lower alkyl sulfonyl; 2-oxo-pyrrolidinyl; lower alkoxy lower alkyl; imidazolyl;
pyrazolyl; and triazolyl;
R₂ is O or S;
R₃ is lower alkyl;
R₄ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen;
quinoxalinyl; or phenyl substituted with alkoxy
R₅ is hydrogen or halogen;
n is 0 or 1;
R₆ is oxido;
with the proviso that if n=1, the N-atom bearing the radical R₆ has a positive charge;
R₇ is hydrogen or amino;
or a tautomer thereof, or a pharmaceutically acceptable salt, or a hydrate or solvate thereof,
(b) at least one non-ionic surfactant that is Vitamin E TPGS in an amount that is about 15 to about 80% by weight of the composition, and
(c) at least one dissolution enhancing agent selected from polyethylene glycol, polyethylene oxide, and any combination of the foregoing.

2. The pharmaceutical composition of claim 1, wherein the therapeutic compound of formula (I) is 2-Methyl-2-[4-(3-methyl-2-oxo-8-quinolin-3-yl-2,3-dihydro-imidazo[4,5-c]quinolin-1-yl)-phenyl]-propionitrile or its monotosylate salt.

3. The pharmaceutical composition according to claim 1, wherein the compound of formula (I) or a pharmaceutically acceptable salt thereof is present in an amount ranging from about 30 to about 60% by weight based on the total weight of the composition.

4. The pharmaceutical composition according to claim 1, wherein the at least one non-ionic surfactant that is Vitamin E TPGS is present in an amount ranging from about 15 to about 45% by weight of the composition.

5. The pharmaceutical composition according to claim 1, wherein the at least one dissolution enhancing agent is present in an amount ranging from about 1 to about 15% by weight of the composition.

6. The pharmaceutical composition of claim 1, wherein the at least one dissolution enhancing agent is polyethylene glycol.

7. The pharmaceutical composition of claim 6, wherein the at least one dissolution enhancing agent is PEG3350.

8. The pharmaceutical composition according to claim 1, wherein the composition is formulated in an oral dosage form, preferably a capsule or a sachet.

9. The pharmaceutical composition according to claim 1, wherein the pharmaceutical composition is a supersaturated system.

10. A process for making the pharmaceutical composition of claim 1 comprising the steps of (a) combining or mixing the therapeutic compound of formula (I) or a tautomer thereof, or a pharmaceutically acceptable salt, or a hydrate or solvate thereof, at least one non-ionic surfactant Vitamin E TPGS in an amount ranging from about 15 to about 80% of the composition, and at least one dissolution enhancing agent selected from the group consisting of polyethylene glycol (PEG), polyethylene oxide, and any combination of the foregoing, (b) granulating the mixture using an extruder or other suitable equipment while heating the mixture to a product temperature below the melting point of the Vitamin E TPGS; and (c) cooling the granules to room temperature.

11. A process for making the pharmaceutical composition of claim 1 comprising the steps of: (a) dividing the non-ionic surfactant Vitamin E TPGS into a first portion and a second portion, (b) combining or mixing the therapeutic compound of formula (I) or a tautomer thereof, or a pharmaceutically acceptable salt, or a hydrate or solvate thereof, the first portion of the non-ionic surfactant Vitamin E TPGS, and the dissolution enhancing agent selected from the group consisting of polyethylene glycol (PEG), polyethylene oxide, and any combination of the foregoing, (c) heating the second portion of the non-ionic surfactant Vitamin E TPGS, (d) granulating the mixture using an extruder or other suitable equipment while slowly adding the second portion of the non-ionic surfactant Vitamin E TPG to the mixture and maintaining a product temperature below about 38° C., and (e) cooling the granules to room temperature.

12. (canceled)

13. (canceled)

14. A kit comprising (a) an oral pharmaceutical composition which comprises granules that comprise a therapeutic compound of formula (I) or a tautomer thereof, or a pharmaceutically acceptable salt, or a hydrate or solvate thereof, at least one non-ionic surfactant that is Vitamin E TPGS in an amount ranging from about 15 to 80% by weight of the composition and at least one dissolution enhancing agent selected from polyethylene glycol, polyethylene oxide, and any combination of the foregoing, and (b) written instructions, wherein such written instructions provide that such oral pharmaceutical composition may be taken between thirty minutes prior to the consumption of food until about two hours after the consumption of food.

15. (canceled)

16. A method of treatment of subject suffering from suffering from a proliferative disease or a mTOR kinase dependent disease comprising administering a therapeutically effective amount of a pharmaceutical composition according to claim 1 to a subject in need thereof.

17. A method according to claim 16, wherein the pharmaceutical composition is administered in an amount sufficient to provide to provide a AUC of about 200 to about 70,000 ng*h/mL of the compound of formula (I), or a tautomer thereof, or a pharmaceutically acceptable salt, or a hydrate or solvate thereof, in the subject's plasma.

18. A method according to claim 16, wherein the pharmaceutical composition is administered to said subject with food and wherein the administration of such pharmaceutical composition with food results in an increase in the bioavailability of the compound of formula (I) or a tautomer thereof, or a pharmaceutically acceptable salt, or a hydrate or solvate thereof, as compared to administration of the pharmaceutical composition to a subject without food.
